(19) United States
(12) Patent Application Publication
(10) Pub. No.: US 2019/0247021 A1
(43) Pub. Date: Aug. 15, 2019

(54) INFORMATION PROCESSING APPARATUS, INFORMATION PROCESSING METHOD, AND NON-TRANSITORY COMPUTER-READABLE MEDIUM

(71) Applicant: CANON KABUSHIKI KAISHA, Tokyo (JP)

(72) Inventors: Shota Yamada, Kawasaki-shi (JP); Taku Inoue, Machida-shi (JP)

(21) Appl. No.: 16/396,554

(22) Filed: Apr. 26, 2019

Related U.S. Application Data
(63) Continuation of application No. PCT/JP2017/044200, filed on Dec. 8, 2017.

(30) Foreign Application Priority Data
Dec. 27, 2016 (JP) 2016-254370

(51) Int. Cl. A61B 8/08; A61B 5/00; G06T 9/00; G06T 3/40

(52) U.S. Cl. CPC A61B 8/5207; A61B 5/0095; G06T 3/40; G06T 9/00; A61B 5/0059

(57) ABSTRACT

An information processing apparatus obtains photoacoustic data generated on a basis of a photoacoustic signal obtained by irradiating a subject with light, obtains compressed data obtained by compressing the photoacoustic data in accordance with a type of the photoacoustic data obtained on a basis of the same photoacoustic signal, and outputs the compressed data to an external apparatus.

### CROSS-REFERENCE TO RELATED APPLICATIONS

This application is a Continuation of International Patent Application No. PCT/JP2017/044200, filed Dec. 8, 2017, which claims the benefit of Japanese Patent Application No. 2016-254370, filed Dec. 27, 2016, both of which are hereby incorporated by reference herein in their entirety.

### TECHNICAL FIELD

The disclosure of the present invention relates to an information processing apparatus, an information processing method, and a program.

### BACKGROUND ART

As a technique for imaging an internal state of a subject in a low invasive manner, research on photoacoustic imaging has been advanced. Information related to a distribution of a sound pressure inside the subject is obtained on the basis of a photoacoustic signal obtained by a photoacoustic imaging apparatus using the photoacoustic imaging. Furthermore, it has been proposed that an absorption coefficient of a substance inside the subject is imaged on the basis of the distribution of the sound pressure, and image of various types representing a substance component ratio inside the subject and information related to a function such as metabolism are obtained.

In recent years, a medical image used for a diagnosis and various information related to the diagnosis have been also computerized. To reduce the data amount of image data, PTL 1 discloses that the image data is compressed by using a compression rate and a compression method which are determined in accordance with a combination of a type of a modality that has performed imaging of a media image and a captured site.

### CITATION LIST

### Patent Literature

PTL 1 Japanese Patent Laid-Open No. 2006-102109

### SUMMARY OF INVENTION

An information processing apparatus according to an embodiment of the present invention includes an obtaining unit configured to obtain photoacoustic data generated on a basis of a photoacoustic signal obtained by irradiating a subject with light, a compression unit configured to obtain compressed data obtained by compressing the photoacoustic data in accordance with a type of the photoacoustic data obtained on a basis of the photoacoustic signal, and an output unit configured to output the compressed data to an external apparatus.

With the information processing apparatus according to the embodiment of the present invention, it is possible to perform the compression in accordance with the type of the photoacoustic data.

Further features of the present invention will become apparent from the following description of exemplary embodiments with reference to the attached drawings.

### BRIEF DESCRIPTION OF DRAWINGS

FIG. 1 illustrates an example of a configuration of a system including an information processing apparatus according to an embodiment of the present invention.

FIG. 2 illustrates an example of a hardware configuration of the information processing apparatus according to the embodiment of the present invention.

FIG. 3 illustrates an example of a functional configuration of the information processing apparatus according to the embodiment of the present invention.

FIG. 4 is a flow chart illustrating an example of processing performed by the information processing apparatus according to the embodiment of the present invention.

FIG. 5 illustrates an example of a configuration of information obtained by the information processing apparatus according to the embodiment of the present invention.

FIG. 6 is a flow chart illustrating an example of the processing performed by the information processing apparatus according to the embodiment of the present invention.

FIG. 7 illustrates an example of processing performed by the information processing apparatus according to the embodiment of the present invention.

FIG. 8 illustrates an example of the processing performed by the information processing apparatus according to the embodiment of the present invention.

FIG. 9 illustrates an example of the processing performed by the information processing apparatus according to the embodiment of the present invention.

FIG. 10 illustrates an example of a configuration of the information obtained by the information processing apparatus according to the embodiment of the present invention.

FIG. 11 is a flow chart illustrating an example of the processing performed by the information processing apparatus according to the embodiment of the present invention.

FIG. 12 illustrates an example of a screen displayed on a display unit by the information processing apparatus according to the embodiment of the present invention.

FIG. 13 illustrates an example of the screen displayed on the display unit by the information processing apparatus according to the embodiment of the present invention.

### DESCRIPTION OF EMBODIMENTS

Hereinafter, embodiments of the present invention will be described with reference to the drawings.

### First Embodiment

In this specification, an acoustic wave generated by expansion caused inside a subject when the subject is irradiated with light will be referred to as a photoacoustic wave. In addition, an acoustic wave transmitted from a transducer or a reflected wave (echo) obtained when the transmitted acoustic wave is reflected inside the subject will be referred to as an ultrasound wave.

As a method of imaging an internal state of a subject in a low invasive manner, photoacoustic imaging attracts attention. In the photoacoustic imaging, a living matter is irradiated with pulsed light generated from a light source, and a photoacoustic wave generated from a living tissue that has absorbed energy of the pulsed light propagated and diffused in the living matter is detected. Data obtained by using the photoacoustic wave including a photoacoustic image that has been imaged by using the photoacoustic wave will be hereinafter referred to as photoacoustic data. According to the photoacoustic imaging, an elastic wave (photoacoustic wave) that is generated when a subject site absorbs energy of the irradiated light and momentarily expands by using a difference in an absorption rate of light energy between the subject site such as a tumor and other tissues is received by the transducer. This detected signal will be hereinafter referred to as a photoacoustic signal. A photoacoustic imaging apparatus can obtain an optical characteristic distribution inside the living matter, in particular, a light energy absorption density distribution, by performing analysis processing of the photoacoustic signal. The photoacoustic data includes data of various types in accordance with an optical characteristic inside the subject. For example, the photoacoustic data includes an absorption coefficient image indicating an absorption density distribution. In addition, an image indicating the presence of biomolecules such as oxygenated hemoglobin, reduced hemoglobin, water, fat, and collagen, a ratio, or the like is generated from the absorption coefficient image. For example, an image related to an oxygen saturation corresponding to an index indicating an oxygen binding state of hemoglobin is obtained on the basis of a ratio between oxygenated hemoglobin and reduced hemoglobin.

As another method of imaging the internal state of the subject in the low invasive manner, an imaging method using the ultrasound wave is widely used. The imaging method using the ultrasound wave is, for example, a method of generating an image on the basis of a time until the ultrasound wave oscillated from the transducer is reflected by a tissue inside the subject in accordance with an acoustic impedance difference and the reflected wave reaches the transducer or an intensity of the reflected wave. The image obtained by the imaging using the ultrasound wave will be hereinafter referred to as an ultrasound image. A user performs an operation by changing an angle of a probe or the like and can observe an ultrasound image in various cross sections at real time. A shape of an organ or a tissue is drawn in the ultrasound image to be utilized for a discovery of a tumor or the like.

To increase an accuracy of a diagnosis, different phenomena at the same site of the subject are imaged on the basis of different principles, and various information may be collected in some cases. An imaging apparatus configured to perform capturing of the ultrasound image and capturing of the photoacoustic image and obtain an image in which respective characteristics are combined with each other has been under review. In particular, since the imaging of not only the ultrasound image but also the photoacoustic image is performed by using the ultrasound wave from the subject, the imaging of the ultrasound image and the imaging of the photoacoustic image can be performed by the same imaging apparatus. More specifically, a configuration can be adopted in which the reflected wave with which the subject is irradiated and the photoacoustic wave are received by the same transducer. With this configuration, an ultrasound signal and a photoacoustic signal can be obtained by the single probe, and it is possible to realize the imaging apparatus that performs the imaging of the ultrasound image and the imaging of the photoacoustic image without complicating the hardware configuration.

In recent years too, a medical image used for a diagnosis and various information related to the diagnosis including the above-described photoacoustic image have been computerized. For example, a Digital Imaging and Communications in Medicine (DICOM) standard is used in many cases for information coordination between an imaging apparatus and various apparatuses connected to the imaging apparatus. The DICOM is the standard for defining formats of the medical images and communication protocols between the apparatuses that deal with those images. Data set as a target to be exchanged on the basis of the DICOM is referred to as information object (IOD: Information Object Definitions). Hereinafter, the information object may be referred to as an IOD or an object in some cases. Examples of the IOD include the medical image, patient information, examination information, structured report, and the like, and various data related to the examination using the medical image and treatment may be set as the target.

The image dealt with on the basis of the DICOM, that is, the image corresponding to the IOD is constituted by meta data and image data. The meta data includes, for example, information related to a patient, an examination, a series, and an image. The meta data is constituted by a set of data elements called DICOM data elements. A tag for identifying the data element is added to each of the DICOM data elements. The image data is pixel data to which a tag indicating the image data is added.

In the photoacoustic imaging, the photoacoustic data of various types can be obtained from the photoacoustic signal related to the single capturing as described above, but when all of the obtained photoacoustic data of the plural types are saved, there is a fear that the capacity of the apparatus for the saving may be squeezed. Furthermore, the imaging apparatus that performs the imaging of the ultrasound image and the imaging of the photoacoustic image can also obtain the ultrasound image at the same time, and it is conceivable that the capacity required for the saving is further increased. In a case where the images of the various types are obtained by the single examination as in the photoacoustic imaging apparatus, it is conceivable that the capacity related to the saving is increased, but when the technology disclosed in PTL 1 is used, the photoacoustic images of the various types which are obtained by the same modality are uniformly compressed, and compression in accordance with a type is not taken into account. According to a first embodiment, to reduce the capacity of the data output to be output to the external apparatus in the imaging apparatus IOD with which it is possible to perform the imaging of the ultrasound image and the imaging of the photoacoustic image, an example will be described according to the first embodiment in which the image data is compressed in accordance with the type of the photoacoustic data.

Configuration of Information Processing Apparatus 107

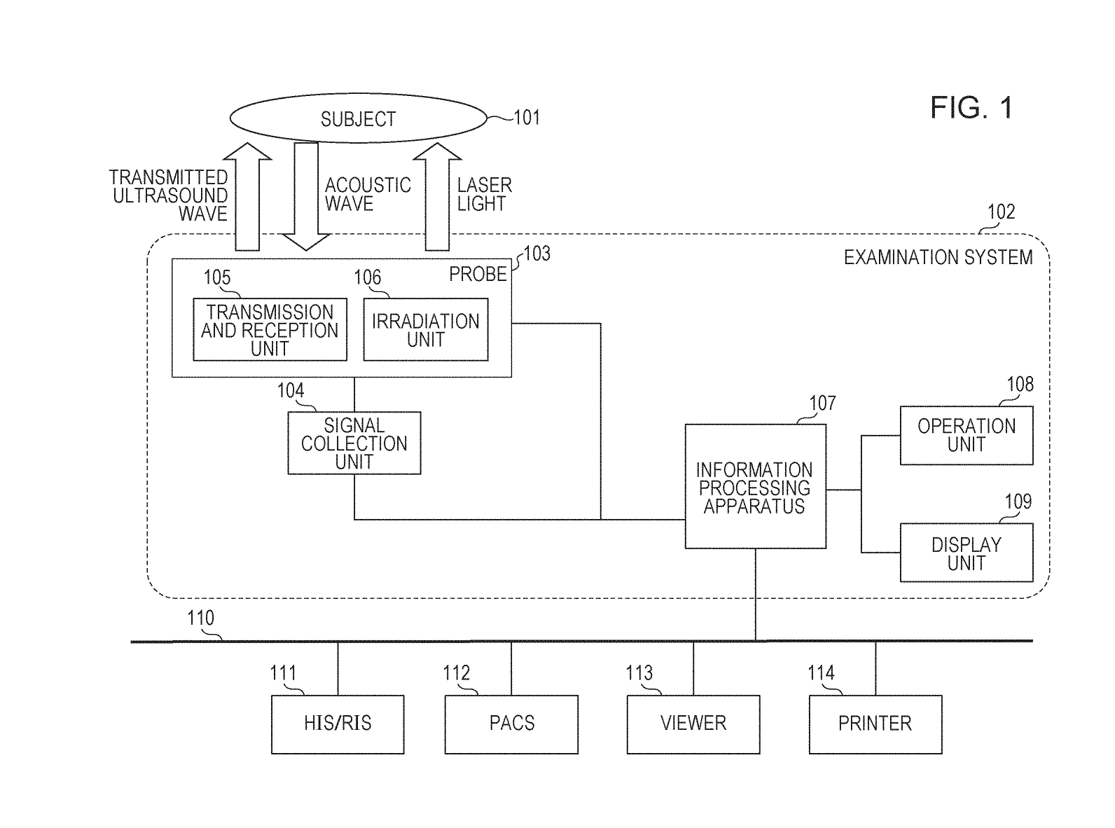

FIG. 1 illustrates an example of a configuration of an examination system 102 including an information processing apparatus 107 according to the first embodiment. The examination system 102 that can generate an ultrasound image and a photoacoustic image is connected to various external apparatuses via a network 110. The respective configurations and various external apparatuses included in the examination system 102 are not required to be installed in the same facility, and it is sufficient when these configurations are connected to one another so as to be mutually communicable.

The examination system 102 includes the information processing apparatus 107, a probe 103, a signal collection unit 104, a display unit 109, and an operation unit 108. The information processing apparatus 107 obtains the information related to the examination including the imaging of the ultrasound image and the imaging of the photoacoustic image from an HIS/RIS 111 and controls the probe 103 and the display unit 109 when the above-described examination is performed. The information processing apparatus 107 obtains the ultrasound signal and the photoacoustic signal from the probe 103 and the signal collection unit 104. The information processing apparatus 107 obtains the ultrasound image on the basis of the ultrasound signal and obtains the photoacoustic image on the basis of the photoacoustic signal. That is, the information processing apparatus 107 obtains the photoacoustic data. The information processing apparatus 107 may further obtain a superimposed image obtained by superimposing the photoacoustic image on the ultrasound image. The information processing apparatus 107 performs transmission and reception of information with an external apparatus such as the HIS/RIS 111 or a PACS 112 in conformity to standards such as Health level 7 (HL7) and Digital Imaging and Communications in Medicine (DICOM).

Regions in a subject 101 in which the imaging of the ultrasound image is performed in the examination system 102 are, for example, regions such as a cardiovascular region, breasts, liver, pancreas, and abdomen. In addition, the imaging of the ultrasound image of the subject who has an administration of an ultrasound contrast agent using microbubbles may be performed in the examination system 102, for example.

In addition, regions in the subject in which the photoacoustic data is imaged in the examination system 102 are, for example, regions such as a cardiovascular region, breasts, neck, abdomen, and extremities including fingers and toes. In particular, a vascular region including plaque of a new blood vessel and a blood vessel wall may also be set as the targets for obtaining the photoacoustic data in accordance with the characteristic related to the light absorption inside the subject. In the examination system 102, for example, the photoacoustic data of the subject 101 who has an administration of a dye such as methylene blue or indocyanine green or small gold particles, or a substance obtained by integrating or chemically modifying those as a contrast agent may also be performed.

The probe 103 is operated by the user and transmits the ultrasound signal and the photoacoustic signal to the signal collection unit 104 and the information processing apparatus 107. The probe 103 includes a transmission and reception unit 105 and an irradiation unit 106. The probe 103 transmits an ultrasound wave from the transmission and reception unit 105 and receives the reflected wave by the transmission and reception unit 105. In addition, the probe 103 irradiates the subject with the light from the irradiation unit 106 and receives the photoacoustic wave by the transmission and reception unit 105. The probe 103 is preferably controlled such that, when information indicating a contact with the subject is received, the transmission of the ultrasound wave for obtaining the ultrasound signal and the light irradiation for obtaining the photoacoustic signal are executed.

The transmission and reception unit 105 includes at least one transducer (not illustrated), a matching layer (not illustrated), a damper (not illustrated), and an acoustic lens (not illustrated). The transducer (not illustrated) is composed of a substance indicating a piezoelectric effect such as lead zirconate titanate (PZT) or polyvinylidene difluoride (PVDF). The transducer (not illustrated) may be an element other than a piezoelectric element and is, for example, a capacitive micro-machined ultrasonic transducer (CMUT) or a transducer using a Fabry-Perot interferometer. Typically, the ultrasound signal is composed of a frequency component at 2 to 20 MHz, and the photoacoustic signal is composed of a frequency component at 0.1 to 100 MHz. The transducer (not illustrated) that can detect these frequencies is used, for example. The signal obtained by the transducer (not illustrated) is a time-resolved signal. An amplitude of the received signal represents a value based on an acoustic pressure received by the transducer at each time. The transmission and reception unit 105 includes a circuit (not illustrated) for an electronic focus or a control unit. An array of the transducers (not illustrated) is, for example, a sector linear array, a convex annular array, or a matrix array. The probe 103 obtains the ultrasound signal and the photoacoustic signal. The probe 103 may alternately obtain the ultrasound signal and the photoacoustic signal, may obtain those signals at the same time, and may also obtain those signals in a previously determined manner.

The transmission and reception unit 105 may be provided with an amplifier (not illustrated) configured to amplify a time-series analog signal received by the transducer (not illustrated). The transducers (not illustrated) may be divided for the transmission and for the reception in accordance with a purpose of the imaging of the ultrasound image. In addition, the transducers (not illustrated) may be divided for the imaging of the ultrasound image and the imaging of the photoacoustic image.

The irradiation unit 106 includes a light source (not illustrated) arranged to obtain the photoacoustic signal and an optical system (not illustrated) arranged to guide the pulsed light emitted from the light source (not illustrated) to the subject. A pulse width of the light emitted from the light source (not illustrated) is, for example, a pulse width higher than or equal to 1 ns and lower than or equal to 100 ns. In addition, a wavelength of the light emitted from the light source (not illustrated) is, for example, a wavelength higher than or equal to 400 nm and lower than or equal to 1600 nm. In a case where the imaging of a blood vessel in the vicinity of a surface of the subject is performed in a high resolution, a wavelength higher than or equal to 400 nm and lower than or equal to 700 nm where the absorption in the blood vessel is large is preferably used. In addition, in a case where the imaging of a deep section of the subject is performed, a wavelength higher than or equal to 700 nm and lower than or equal to 1100 nm where the absorption hardly occurs in a tissue such as water or fat is preferably used.

The light source (not illustrated) is laser or a light emitting diode, for example. The irradiation unit 106 may also use a light source that can convert a wavelength to obtain the photoacoustic signal by using the light at a plurality of wavelengths. As an alternative to the above-described configuration, a configuration may be adopted in which the irradiation unit 106 is provided with a plurality of light sources configured to generate light having mutually different wavelengths and can emit the light having the mutually different wavelengths from the respective light sources. The laser is, for example, solid laser, gas laser, dye laser, or semiconductor laser. Pulsed laser such as Nd:YAG laser or alexandrite laser may be used as the light source (not illustrated). In addition, Ti:sa laser or optical parametric oscillators (OPO) laser in which light of the Nd:YAG laser is set as excitation light may be used as the light source (not illustrated). In addition, a microwave source may be used as the light source (not illustrated).

An optical element such as a lens, a mirror, or an optical fiber is used as the optical system (not illustrated). In a case where the subject is breast, since the irradiation is preferably performed by increasing a beam diameter of the pulsed light, the optical system (not illustrated) may also be provided with a diffused plate that diffuses the emitted light. As an alternative to the above-described configuration, a configuration may be adopted in which the optical system (not illustrated) is provided with a lens or the like and can focus beam to increase the resolution.

The signal collection unit 104 respectively converts a reflected wave received by the probe 103 and an analog signal related to the photoacoustic wave into digital signals. The signal collection unit 104 transmits the ultrasound signal and the photoacoustic signal which have been converted into the digital signals to the information processing apparatus 107.

The display unit 109 displays the image obtained by the imaging in the examination system 102 and the information related to the examination on the basis of the control from the information processing apparatus 107. The display unit 109 provides an interface configured to accept an instruction of the user on the basis of the control from the information processing apparatus 107. The display unit 109 is, for example, a liquid crystal display.

The operation unit 108 transmits the information related to the operation input of the user to the information processing apparatus 107. The operation unit 108 is, for example, a key board or a track ball or various buttons for performing operation inputs related to the examination.

It should be noted that the display unit 109 and the operation unit 108 may also be integrated with each other as a touch panel display. In addition, the information processing apparatus 107, the display unit 109, and the operation unit 108 are not required to be separate apparatuses and may be realized as a console in which these configurations are integrated with one another. The information processing apparatus 107 may also include a plurality of probes.

The HIS/RIS 111 is a system for managing patient information and examination information. The hospital information system (HIS) is a system for assisting operations in a hospital. The HIS includes an electronic medical record system, an ordering system, and a medical accounting system. The radiology information system (RIS) is a system for managing examination information in a radiology department and managing progresses of the respective examinations in the imaging apparatus. The examination information includes an examination ID for uniquely identifying the examination and information related to a capturing technique included in the above-described examination. An ordering system constructed for each department may be connected to the examination system 102 instead of the RIS or in addition to the RIS. A procedure from an examination order issuance to accounting is managed in coordination with one another by the HIS/RIS 111. The HIS/RIS 111 transmits the information of the examination performed by the examination system 102 to the information processing apparatus 107 in accordance with a query from the information processing apparatus 107. The HIS/RIS 111 receives information related to the progress of the examination from the information processing apparatus 107. When the HIS/RIS 111 receives information indicating that the examination has been completed from the information processing apparatus 107, the HIS/RIS 111 performs processing for accounting.

The picture archiving and communication system (PACS) 112 is a database system where images obtained by various imaging apparatuses inside or outside the facility are held. The PACS 112 includes a storage unit (not illustrated) configured to store a medical image and auxiliary information such as a capturing condition of the medical image, a parameter of image processing including reconstruction, and patient information and a controller (not illustrated) configured to manage the information stored in the storage unit. The PACS 112 stores an ultrasound image, a photoacoustic image, or a superimposed image corresponding to an object that has been output from the information processing apparatus 107. The communication between the PACS 112 and the information processing apparatus 107 and the images stored in the PACS 112 are preferably in conformity to the standards such as the HL7 and the DICOM. The various images output from the information processing apparatus 107 are stored while the auxiliary information is associated with various tags in conformity to the DICOM standard.

A viewer 113 is a terminal for an image diagnosis and reads out the image stored in the PACS 112 or the like to be displayed for the diagnosis. A doctor displays the image on the viewer 113 to observe and records information obtained as a result of the observation as an image diagnosis report. The image diagnosis report created by using the viewer 113 may be stored in the viewer 113 or output to the PACS 112 or a report server (not illustrated) to be stored.

A printer 114 prints the image stored in the PACS 112 or the like. The printer 114 is a film printer, for example, and prints the image stored in the PACS 112 or the like on a film to be output.

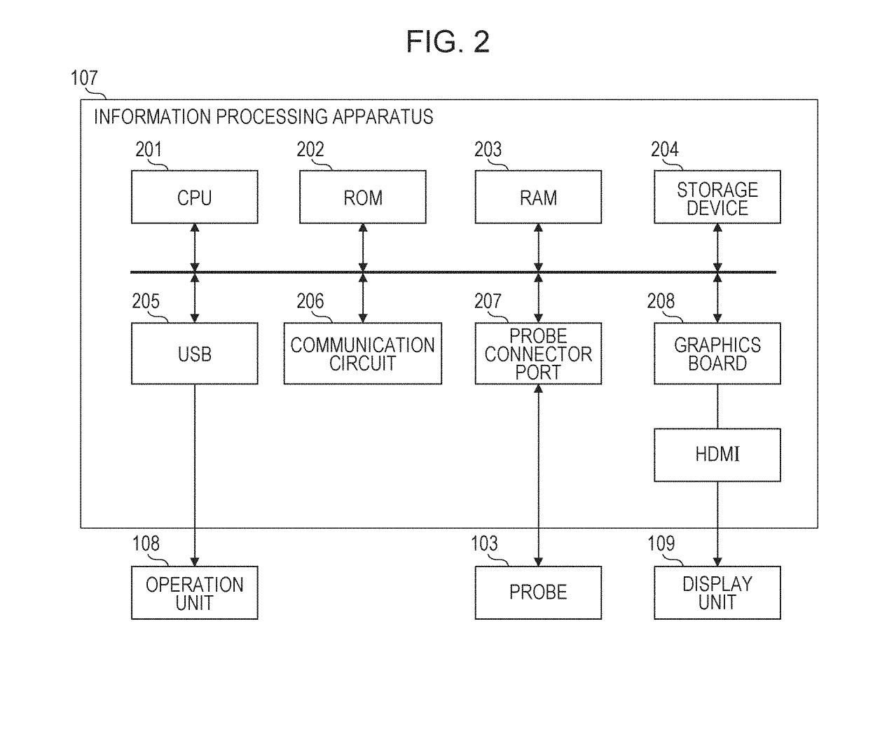

FIG. 2 illustrates an example of a hardware configuration of the information processing apparatus 107. The information processing apparatus 107 is a computer, for example. The information processing apparatus 107 includes a CPU 201, a ROM 202, a RAM 203, a storage device 204, a universal serial bus (USB) 205, and a communication circuit 206, a probe connector port 207, and a graphics board 208. These components are connected to one another via a BUS so as to be mutually communicable. The BUS is used for transmission and reception of data between connected hardware and transmission of a command from the CPU 201 to the other hardware.

The central processing unit (CPU) 201 is a control circuit configured to control the information processing apparatus 107 and the respective units connected to the information processing apparatus 107 in an integrated manner. The CPU 201 implements control by executing a program stored in the ROM 202. The CPU 201 also executes a display driver corresponding to software configured to control the display unit 109 and performs display control with respect to the display unit 109. Furthermore, the CPU 201 performs input and output control with respect to the operation unit 108.

The read only memory (ROM) 202 stores a program that stores a procedure of the control by the CPU 201 and data. The ROM 202 stores a boot program of the information processing apparatus 107 and various pieces of initial data. In addition, the ROM 202 stores various programs for realizing the processing of the information processing apparatus 107.

The random access memory (RAM) 203 is configured to provide a working storage area when control based on a command program is performed by the CPU 201. The RAM 203 includes a stack and a work area. The RAM 203 stores programs for executing the processes in the information processing apparatus 107 and the respective units connected to the information processing apparatus 107 and various parameters used in the image processing. The RAM 203 stores a control program to be executed by the CPU 201 and temporarily stores various pieces of data when the CPU 201 performs various types of control.

The storage device 204 is an auxiliary storage device configured to save various pieces of data such as the ultrasound image and the photoacoustic data including the photoacoustic image. The storage device 204 is, for example, a hard disk drive (HDD) or a solid state drive (SSD).

The universal serial bus (USB) 205 is a connection unit to which the operation unit 108 is connected.

The communication circuit 206 is a circuit configured to perform communications with the respective units that constitute the examination system 102 and various external apparatuses connected to the network 110. The communication circuit 206 stores the information to be output in a transfer packet and performs the output to the external apparatus via the network 110 by a communication technology such as TCP/IP, for example. The information processing apparatus 107 may also include a plurality of communication circuits in accordance with a desired communication mode.

The probe connector port 207 is a connection opening for connecting the probe 103 to the information processing apparatus 107.

The graphics board 208 includes a graphics processing unit (GPU) and a video memory. The GPU performs a calculation related to reconstruction processing for generating the photoacoustic image from the photoacoustic signal, for example.

High-Definition Multimedia Interface (HDMI) (registered trademark) 209 is a connection unit to which the display unit 109 is connected.

The CPU 201 or the GPU is an example of a processor. In addition, the ROM 202, the RAM 203, or the storage device 204 is an example of a memory. The information processing apparatus 107 may include a plurality of processors. According to the first embodiment, when the processor of the information processing apparatus 107 executes the programs stored in the memory, the functions of the respective units of the information processing apparatus 107 are realized.

In addition, the information processing apparatus 107 may also include a CPU, a GPU, or an application specific integrated circuit (ASIC) that dedicatedly performs particular processing. The information processing apparatus 107 may also include a field-programmable gate array (FPGA) in which particular processing or all processes are programmed.

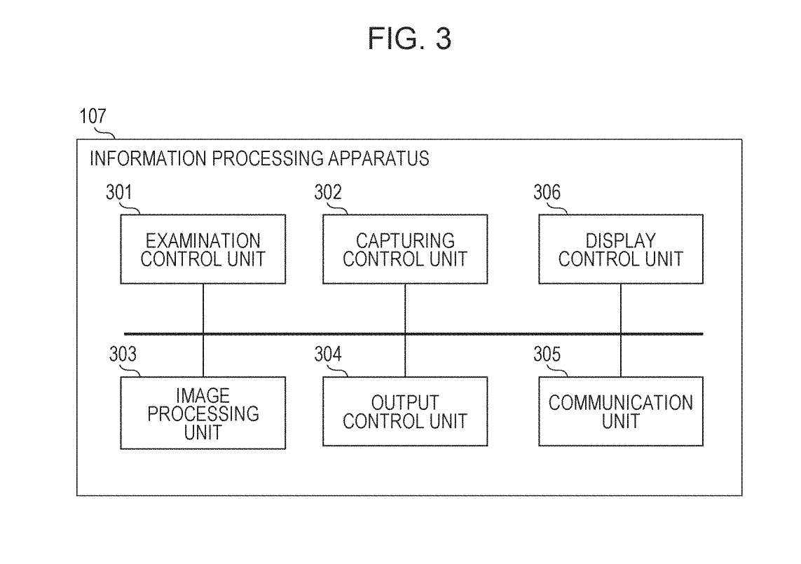

FIG. 3 illustrates an example of a functional configuration of the information processing apparatus 107. The information processing apparatus 107 includes an examination control unit 301, a capturing control unit 302, an image processing unit 303, an output control unit 304, a communication unit 305, and a display control unit 306.

The examination control unit 301 obtains the information of the examination order from the HIS/RIS 111. The examination order includes the information of the patient subjected to the examination and the information related to the capturing technique. The examination control unit 301 transmits the information related to the examination order to the capturing control unit 302. In addition, the examination control unit 301 causes the display unit 109 to display the information of the above-described examination such that the information related to the examination is presented to the user via the display control unit 306. The information of the examination displayed on the display unit 109 includes the information of the patient subjected to the examination, the information of the capturing technique included in the above-described examination, and the image generated when the imaging has been already completed. The examination control unit 301 further transmits the information related to the progress of the above-described examination to the HIS/RIS 111 via the communication unit 305.

The capturing control unit 302 controls the probe 103 on the basis of the information of the capturing technique received from the examination control unit 301 and obtains the ultrasound signal and the photoacoustic signal from the probe 103 and the signal collection unit 104. The capturing control unit 302 instructs the irradiation unit 106 to perform the light irradiation. The capturing control unit 302 instructs the transmission and reception unit 105 to perform the transmission of the ultrasound wave. The capturing control unit 302 executes the instruction to the irradiation unit 106 and the instruction to the transmission and reception unit 105 on the basis of the operation input of the user and the information of the capturing technique. The capturing control unit 302 also instructs the transmission and reception unit 105 to perform the reception of the ultrasound wave. The capturing control unit 302 instructs the signal collection unit 104 to perform the signal sampling. The capturing control unit 302 controls the probe 103 as described above and obtains the ultrasound signal and the photoacoustic signal while being distinguished from each other. In addition, the capturing control unit 302 obtains information related to timings when the ultrasound signal and the photoacoustic signal are obtained (hereinafter, referred to as timing information). The timing information refers, for example, to information indicating the timing for the light irradiation or the transmission of the ultrasound wave when the capturing control unit 302 controls the probe 103. The information indicating the timing may be a time or an elapsed time since the examination is started. It should be noted that the capturing control unit 302 obtains the ultrasound signal and the photoacoustic signal converted into the digital signals output from the signal collection unit 104.

The image processing unit 303 generates the ultrasound image and the photoacoustic image. That is, the image processing unit 303 obtains the photoacoustic data. In addition, the compressed image (compressed data) in which the ultrasound image or the photoacoustic image (photoacoustic data) is compressed is generated in accordance with the control from the output control unit 304. Furthermore, the image processing unit 303 may generate the superimposed image obtained by superimposing the photoacoustic image on the ultrasound image. In addition, the image processing unit 303 may generate a moving picture composed of the ultrasound image and the photoacoustic image.

Specifically, the image processing unit 303 generates the photoacoustic data on the basis of the photoacoustic signal obtained by the capturing control unit 302. The image processing unit 303 reconstructs a distribution of the acoustic wave when the light irradiation is performed on the basis of the photoacoustic signal (which will be hereinafter referred to as an initial sound pressure distribution, and the data related to the initial sound pressure distribution will be referred to as initial sound pressure data). The image processing unit 303 obtains an absorption coefficient distribution of the light in the subject by dividing the reconstructed initial sound pressure distribution by a light fluence distribution of the subject with regard to the light with which the subject is irradiated. In addition, by using a state in which a light absorption degree in the subject varies in accordance with the wavelength of the light with which the subject is irradiated, a density distribution of a substance in the subject is obtained from the absorption coefficient distribution with respect to the plurality of wavelengths. For example, the image processing unit 303 obtains density distributions of substances in the subject with regard to oxyhemoglobin and deoxyhemoglobin. The image processing unit 303 further obtains an oxygen saturation distribution as a ratio of an oxyhemoglobin density with respect to a deoxyhemoglobin density. The photoacoustic data generated by the image processing unit 303 is, for example, data or an image indicating at least one piece of information including the above-described initial sound pressure distribution, the light fluence distribution, the absorption coefficient distribution, the substance density distribution, and the oxygen saturation distribution.

In addition, the image processing unit 303 obtains an emission line in which an amplitude of the reflected wave of the ultrasound signal is converted into a luminance and changes a display position of the emission light in accordance with scanning of ultrasound beam to generate an ultrasound image (B-mode image). In a case where the probe 103 is a three-dimensional probe, the image processing unit 303 can generate an ultrasound image (C-mode image) composed of orthogonal three cross sections. The image processing unit 303 generates an arbitrary cross section or a stereoscopic image after rendering on the basis of the three-dimensional ultrasound image. The image processing unit 303 is an example of an image obtaining unit configured to obtain the ultrasound image and the photoacoustic image (photoacoustic data).

The image processing unit 303 generates a compressed image (compressed data) of the ultrasound image or the photoacoustic image (photoacoustic data) in accordance with the control from the output control unit 304. The image processing unit 303 performs compression processing on the image data to be compressed in accordance with a type thereof and generates the compression data. The image processing unit 303 can compress the image data by various methods such as, for example, entropy coding, run length compression, Joint Photographic Experts Group (JPEG) compression, and wavelet compression. In addition, the image processing unit 303 can compress the image data by techniques exemplified in FIG. 7 to FIG. 9. A detail of the processing related to the compression will be described below.

The output control unit 304 generates an object for transmitting various information to an external apparatus such as the PACS 112 or the viewer 113 in accordance with the control from the examination control unit 301 or the operation input of the user. The object refers to information set as a target to be transmitted from the information processing apparatus 107 to the external apparatus such as the PACS 112 or the viewer 113. For example, the output control unit 304 generates an IOD for outputting the ultrasound image and the photoacoustic image generated by the image processing unit 303 to the PACS 112.

The output control unit 304 controls the image processing unit 303 so as to compress the image data to be output as the IOD in accordance with a predetermined setting or the operation input of the user. The output control unit 304 controls the processing related to the compression in accordance with the types of the photoacoustic image (photoacoustic data) and the ultrasound image to be output.

The object to be output to the external apparatus includes the auxiliary information added as various tags in conformity to the DICOM standard. The auxiliary information includes, for example, patient information, information indicating the imaging apparatus that has performed the imaging of the above-described image, an image ID for uniquely identifying the above-described image, an examination ID for uniquely identifying the examination in which the imaging of the above-described image has been performed, and information of the probe 103. The auxiliary information of the IOD related to the compression data includes the information related to the compression of the above-described compression data. The information related to the compression refers to, for example, information related to a method of the compression processing and decoding of the above-described compression data. In addition, the auxiliary information generated by the output control unit 304 includes information for associating the ultrasound image and the photoacoustic data imaged in the examination with each other.

The communication unit 305 controls the transmission and reception of the information between the external apparatus such as the HIS/RIS 111, the PACS 112, or the viewer 113 and the information processing apparatus 107 via the network 110. A transmission and reception control unit receives the information of the examination order from the HIS/RIS 111. The transmission and reception control unit transmits an object generated by an imaging failure processing control unit to the PACS 112 or the viewer 113.

The display control unit 306 controls the display unit 109 to display the information on the display unit 109. The display control unit 306 causes the display unit 109 to display the information in accordance with an input from another module or the operation input of the user via the operation unit 108. The display control unit 306 is an example of a display control unit.

Series of Processes by Information Processing Apparatus 107

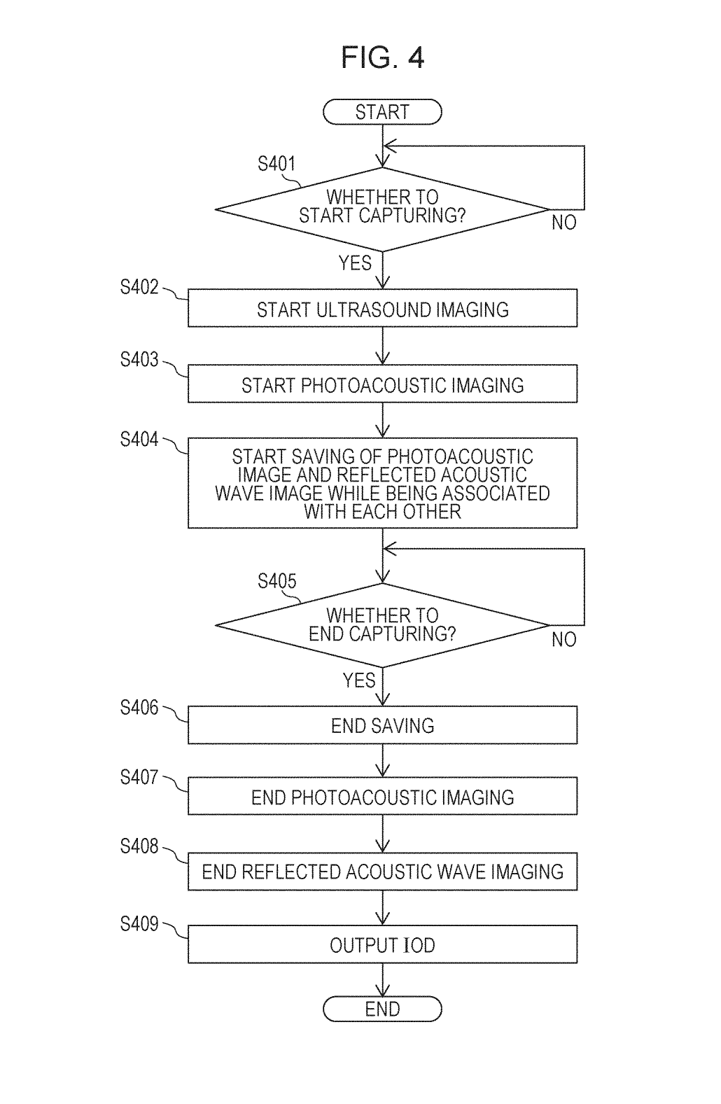

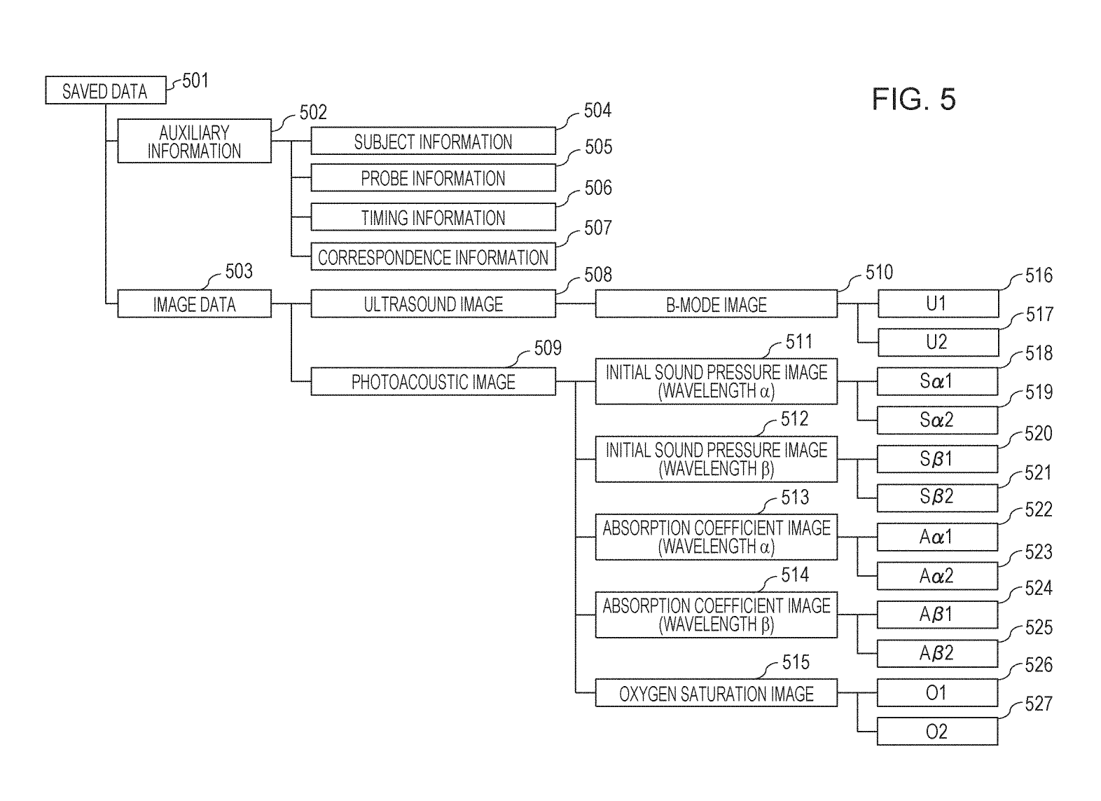

FIG. 4 is a flow chart illustrating an example of processing for the information processing apparatus 107 to obtain the ultrasound image and the photoacoustic image (photoacoustic data) and output the IOD to the external apparatus. In the following processing, unless specifically stated, the main body that realizes the respective processes is the CPU 201 or the GPU. In addition, the information obtained by the information processing apparatus 107 will be described accordingly with reference to FIG. 5.

In step S401, the capturing control unit 302 determines whether or not the capturing is to be started. First, the examination control unit 301 obtains the information of the examination order by the HIS/RIS 111 and transmits the information of the examination order to the capturing control unit 302. The display control unit 306 causes the display unit 109 to display a user interface for the user to input the information of the examination indicated by the above-described examination order and the instruction with respect to the above-described examination. The capturing control unit 302 determines that the capturing is to be started in accordance with the instruction for starting the capturing which has been input to the user interface via the operation unit 108. When the capturing is started, the flow proceeds to step S402.

In step S402, the capturing control unit 302 controls the probe 103 and the signal collection unit 104 to start the imaging of the ultrasound image. The user pushes the probe 103 against the subject 101 to perform the imaging at a desired position. The capturing control unit 302 obtains the ultrasound signal corresponding to the digital signal and the timing information related to the obtainment of the above-described ultrasound signal to be stored in the RAM 203. The image processing unit 303 generates the ultrasound image by performing processing such as phasing addition (delay and sum) with respect to the ultrasound signal. It should be noted that the ultrasound signal saved in the RAM 203 may be deleted when the ultrasound image is generated. The image processing unit 303 causes the display unit 109 to display the obtained ultrasound image via the display control unit 306. The capturing control unit 302 and the image processing unit 303 repeatedly execute these steps to update the ultrasound image displayed on the display unit 109. With this configuration, the ultrasound image is displayed as a moving picture.

In step S403, the capturing control unit 302 controls the probe 103 and the signal collection unit 104 to start the imaging of the photoacoustic image. The user pushes the probe 103 against the subject 101 to perform the imaging at a desired position. The capturing control unit 302 obtains the photoacoustic signal corresponding to the digital signal and the timing information related to the obtainment of the above-described photoacoustic signal to be stored in the RAM 203. The image processing unit 303 generates the photoacoustic data by performing processing such as universal back-projection (UBP) with respect to the photoacoustic signal. It should be noted that the photoacoustic signal saved in the RAM 203 may be deleted when the photoacoustic data is generated. The image processing unit 303 causes the display unit 109 to display the obtained photoacoustic data via the display control unit 306. The capturing control unit 302 and the image processing unit 303 repeatedly execute these steps to update the photoacoustic data displayed on the display unit 109. With this configuration, the photoacoustic data is displayed as a moving picture.

The processing in step S402 and the processing in step S403 may be performed at the same time, may be switched at every predetermined interval, or may be switched on the basis of the operation input of the user or the examination order. The example in which the imaging of the ultrasound image is performed earlier has been described, but the imaging of the photoacoustic image may be performed earlier. In the display control unit 306, when the ultrasound image and the photoacoustic image are to be displayed in step S402, one of the images may be superimposed on the other image to be displayed, or those images may be displayed next to each other. In addition, the image processing unit 303 may obtain the superimposed image obtained by superimposing the ultrasound image and the photoacoustic image on each other, and the display control unit 306 may cause the display unit 109 to display the superimposed image.

In step S404, the output control unit 304 associates the ultrasound image and the photoacoustic image (photoacoustic data) obtained in step S402 and step S403 with each other to be stored in the storage device 204 together with the auxiliary information together with the auxiliary information. In step S404, the output control unit 304 repeatedly performs the processing with respect to the ultrasound image and the photoacoustic image of the respective frames obtained in step S402 and step S403 so that those can be saved as a file including the ultrasound image and the photoacoustic image. The output control unit 304 starts the processing related to the saving in accordance with an operation input for instructing to capture a still image or an operation input for instructing to start to capture a moving picture.

FIG. 5 illustrates an example of a structure of data where saving is to be started in step S404. Saved data 501 includes auxiliary information 502 and image data 503. The auxiliary information 502 may be recorded in a header part of the saved data 501.

The auxiliary information 502 includes, for example, subject information 504, probe information 505, timing information 506, and correspondence information 507.

The subject information 504 is information related to the subject 101. The subject information 504 includes at least one piece of information such as, for example, subject ID, subject name, age, blood pressure, heart rate, body temperature, height, weight, pre-existing condition, gestational age, and examination information. It should be noted that, in a case where the examination system 102 includes an electrocardiograph (not illustrated) or a pulse oximeter (not illustrated), information such as an electrocardiogram or an oxygen saturation may be saved as the subject information 504.

The probe information 505 is information related to the probe 103 used in the capturing. The probe information 505 includes the information related to the probe 103 such as a type of the probe 103 and a position and an inclination at the time of the imaging. The examination system 102 may be provided with a magnetic sensor (not illustrated) that detects the position and the inclination of the probe 103, and the capturing control unit 302 may also obtain these pieces of information from the magnetic sensor (not illustrated).

The timing information 506 is information related to a timing when the image data 503 is obtained. The timing information 506 is obtained in step S402 and step S403. The timing information is indicated, for example, by the time or the elapsed time since the examination is started as described above. The timing information of the ultrasound image is information related to a timing when the ultrasound signal used for the above-described ultrasound image is obtained. The timing information in a case where the plurality of ultrasound signals are used for the single ultrasound image may be information related to a timing when an arbitrary ultrasound signal is obtained, and the operation may be unified for the respective ultrasound images obtained in the single examination. The timing when the ultrasound signal is obtained may be a timing when the information processing apparatus 107 receives the ultrasound signal, a timing when the probe 103 transmits the ultrasound wave to the subject 101, a timing when the probe 103 receives the ultrasound wave, a timing when the drive signal of the transmission and reception of the ultrasound wave with respect to the probe 103 is detected, or a timing when the signal collection unit 104 receives the ultrasound signal. The timing information of the photoacoustic data is the information related to a timing when the photoacoustic signal used for the photoacoustic data is obtained. The timing information in a case where the plurality of photoacoustic signals are used for the single photoacoustic data is information related to a timing when an arbitrary photoacoustic signal is obtained, and the operation may be unified for the respective pieces of photoacoustic data obtained in the single examination. The timing when the photoacoustic signal is obtained may be a timing when the information processing apparatus 107 receives the photoacoustic signal, a timing when the probe 103 irradiates the subject 101 with light, a timing when the probe 103 receives the photoacoustic wave, a timing when the drive signal with respect to the probe 103 of the light irradiation or the reception of the photoacoustic wave is detected, or a timing when the signal collection unit 104 receives the photoacoustic signal.

The correspondence information 507 is information that associates ultrasound images 516 and 517 and photoacoustic images 518 to 527 included in the image data 503 with one another. The correspondence information 507 is, for example, information for associating a certain ultrasound image and the photoacoustic image obtained substantially at the same time with each other. In addition, the correspondence information 507 is, for example, information for associating the photoacoustic images of the plural types obtained from the same photoacoustic signal with each other.

The image data 503 includes the ultrasound images 516 and 517 and the photoacoustic images 518 to 527 obtained in step S402 and step S403. The image data 503 includes the ultrasound image and the photoacoustic image obtained substantially at the same time at a certain timing. The image data 503 may include the ultrasound image and the photoacoustic image obtained in the single examination. In addition, the image data 503 may include the ultrasound image and the photoacoustic image in the respective frames constituting the moving picture.

In the example illustrated in FIG. 5, an ultrasound image 508 includes a B-mode image 510 as a type thereof. The B-mode image 510 includes the ultrasound images 516 and 517 to which identifiers U1 and U2 for respectively uniquely identifying are added.

In addition, In the example illustrated in FIG. 5, a photoacoustic image 509 includes the image data based on the photoacoustic signal obtained when the subject is irradiated with light at a wavelength α and the image data based on the photoacoustic signal obtained when the subject is irradiated with light at a wavelength β. Types of the photoacoustic image 509 include an initial sound pressure image (initial sound pressure data) 511 at the wavelength α, an absorption coefficient image 513, an initial sound pressure image (initial sound pressure data) 512 at the wavelength β, an absorption coefficient image 514, and an oxygen saturation image 515. The initial sound pressure image (initial sound pressure data) (wavelength α) 511 includes the photoacoustic images 518 and 519 to which identifiers Sα1 and Sα2 are added to respectively uniquely identify. The initial sound pressure image (initial sound pressure data) (wavelength β) 512 includes the photoacoustic images 520 and 521 to which identifiers Sβ1 and Sβ2 are added to respectively uniquely identify. The absorption coefficient image (wavelength α) includes the photoacoustic images 522 and 523 to which identifiers Aα1 and Aα2 are added to respectively uniquely identify. The absorption coefficient image (wavelength β) includes the photoacoustic images 524 and 525 to which identifiers Aβ1 and Aβ2 are added to respectively uniquely identify. The oxygen saturation image 515 includes the photoacoustic images 526 and 527 to which identifiers O1 and O2 are added to respectively uniquely identify.

In the example illustrated in FIG. 5, the B-mode image U1 and the initial sound pressure image (initial sound pressure data) (wavelength α) Sα1, the initial sound pressure image (initial sound pressure data) (wavelength β) Sβ1, the absorption coefficient image (wavelength α) Aα1, the absorption coefficient image (wavelength β) Aβ1, and the oxygen saturation image O1 are associated with one another by the correspondence information 507. The absorption coefficient image (wavelength α) Aα1 is obtained on the basis of the initial sound pressure image (initial sound pressure data) (wavelength α) Sα1. In addition, the absorption coefficient image (wavelength β) Aβ1 is obtained on the basis of the initial sound pressure image (initial sound pressure data) (wavelength β) Sβ1. The oxygen saturation image O1 is obtained on the basis of the absorption coefficient image (wavelength α) Aα1 and the absorption coefficient image (wavelength β) Aβ1.

In addition, In the example illustrated in FIG. 5, the B-mode image U2 and the initial sound pressure image (initial sound pressure data) (wavelength α) Sα2, the initial sound pressure image (initial sound pressure data) (wavelength β) Sβ2, the absorption coefficient image (wavelength α) Aα2, the absorption coefficient image (wavelength β) Aβ2, and the oxygen saturation image O2 are associated with one another by the correspondence information 507. The absorption coefficient image (wavelength α) Aα2 is obtained on the basis of the initial sound pressure image (initial sound pressure data) (wavelength α) Sα2. In addition, the absorption coefficient image (wavelength β) Aβ2 is obtained on the basis of the initial sound pressure image (initial sound pressure data) (wavelength β) Sβ2. The oxygen saturation image O2 is obtained on the basis of the absorption coefficient image (wavelength α) Aα2 and the absorption coefficient image (wavelength β) Aβ2.

In step S405, the capturing control unit 302 determines whether or not the capturing is to be ended. During the examination, the display control unit 306 causes the display unit 109 to display the user interface for the user to input the instruction. The capturing control unit 302 determines that the capturing is ended on the basis of the instruction for ending the capturing which has been input to the user interface via the operation unit 108. In addition, the capturing control unit 302 may determine that the capturing is ended when a predetermined time has elapsed since the instruction for starting the capturing accepted in step S401 is issued. When the capturing is ended, the examination control unit 301 transmits information indicating that the above-described capturing is ended to the HIS/RIS 111 via the communication unit 305. When the capturing is ended, the flow proceeds to step S406.

In step S406, the capturing control unit 302 controls the probe 103 and the signal collection unit 104 to end the imaging of the photoacoustic image. In step S407, the capturing control unit 302 controls the probe 103 and the signal collection unit 104 to end the imaging of the ultrasound image.

In step S408, the output control unit 304 ends the processing related to the saving of the ultrasound image and the photoacoustic image which has started in step S404.

In step S409, the communication unit 305 outputs the IOD based on the data saved up to step S408 to the external apparatus. The output control unit 304 generates the IOD including the ultrasound image and the photoacoustic image (photoacoustic data) obtained in step S402 and step S403 on the basis of the information saved up to step S407. The communication unit 305 outputs the IOD to the external apparatus such as the PACS 112.

According to the first embodiment, a case where the image data of the IOD output to the external apparatus in step S409 is compressed in accordance with a type thereof will be described as an example.

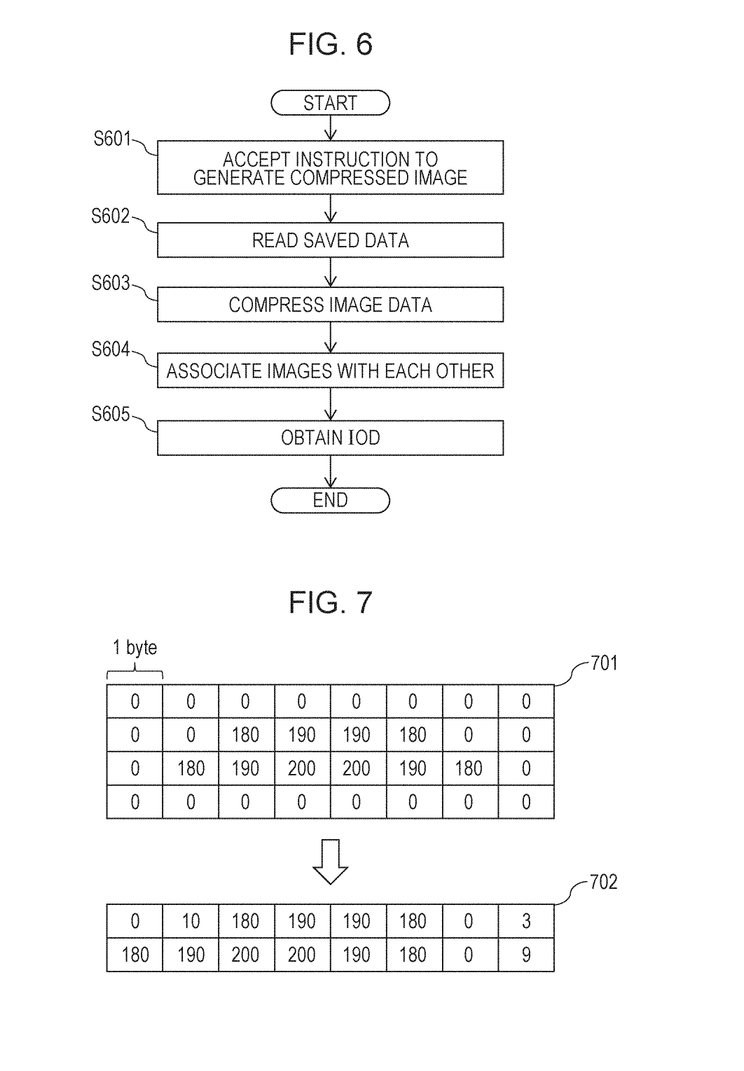

FIG. 6 is a flow chart illustrating an example of processing for the output control unit 304 to compress the image data to be output as the IOD. The series of processes illustrated in FIG. 6 is performed as a sub routine of step S409, for example. In the following processing, unless specifically stated, the main body that realizes the respective processes is the CPU 201 or the GPU.

In step S601, the output control unit 304 controls the image processing unit 303 to start the generation of the compression data. Hereinafter, an example will be described in which the ultrasound imaging and the photoacoustic imaging for two frames are performed, and in the respective frames, the B-mode images as the type of the ultrasound image, the initial sound pressure image (initial sound pressure data) as the type of the photoacoustic image (photoacoustic data), and the image data including the respective absorption coefficient images at the two different wavelengths and the oxygen saturation image are generated. The type of the image data to be output to the external apparatus and whether or not to perform the compression processing with regard to the respective types may be selected on the basis of a predetermined setting.

In step S602, the output control unit 304 reads out the saved data 501 saved in the storage device 204 in step S404 to step S406.

In step S603, the image processing unit 303 generates the compression data in accordance with the type of the image data on the basis of the control by the output control unit 304 in step S601. The image processing unit 303 may generate the compression data by combining a plurality of method with each other.

Here, an example of the compression processing performed by the image processing unit 303 will be described. The image processing unit 303 generates the compressed image (compressed data) by gradation conversion, for example. When one pixel originally represented by 8-bit 256 gradations is converted into one pixel represented by 6-bit 64 gradations, the data amount can be reduced.

In another example, the image processing unit 303 generates the compressed image (compressed data) by reducing the information amount of the high frequency component. A medical image is constituted by a locally moderate change in a pixel value, and it is predicted that a spatial frequency is mainly a low frequency component. For example, the image processing unit 303 uses a discrete cosine transfer (DCT) to convert the image data into a plurality of frequency components (DCT coefficient). The image processing unit 303 reduces the information amount of the high frequency component by dividing the DCT coefficient by a quantization table.

FIG. 7 illustrates an example of lossless compression performed by the image processing unit 303 with respect to the photoacoustic image. The image processing unit 303 generates the compressed image (compressed data) on the basis of a run length in which a particular pixel value is continuous. In the photoacoustic image, for example, the light emitted from the probe 103 may not reach a deep part of the subject 101 in some cases. Therefore, it is conceivable that a large number of pixels where the pixel value is 0 which does not include the information of the subject 101 exist in the photoacoustic image. With regard to the compressed image 702, when 0 is stored in the first byte and the number of continuous pixels where the pixel value of the original image 701 is 0 is stored in the second byte, the information amount is reduced.

The IOD of the image data compressed by the processing exemplified in FIG. 7 is output together with the information indicating that the compression is performed on the basis of the run length in which the particular pixel value is continuous. The external apparatus such as the viewer 113 that has obtained the IOD previously obtains the information for decoding the compression data obtained by the above-described compression method. The viewer 113 reads out the information indicating the above-described compression method from the IOD, so that it is possible to decode the compression data.

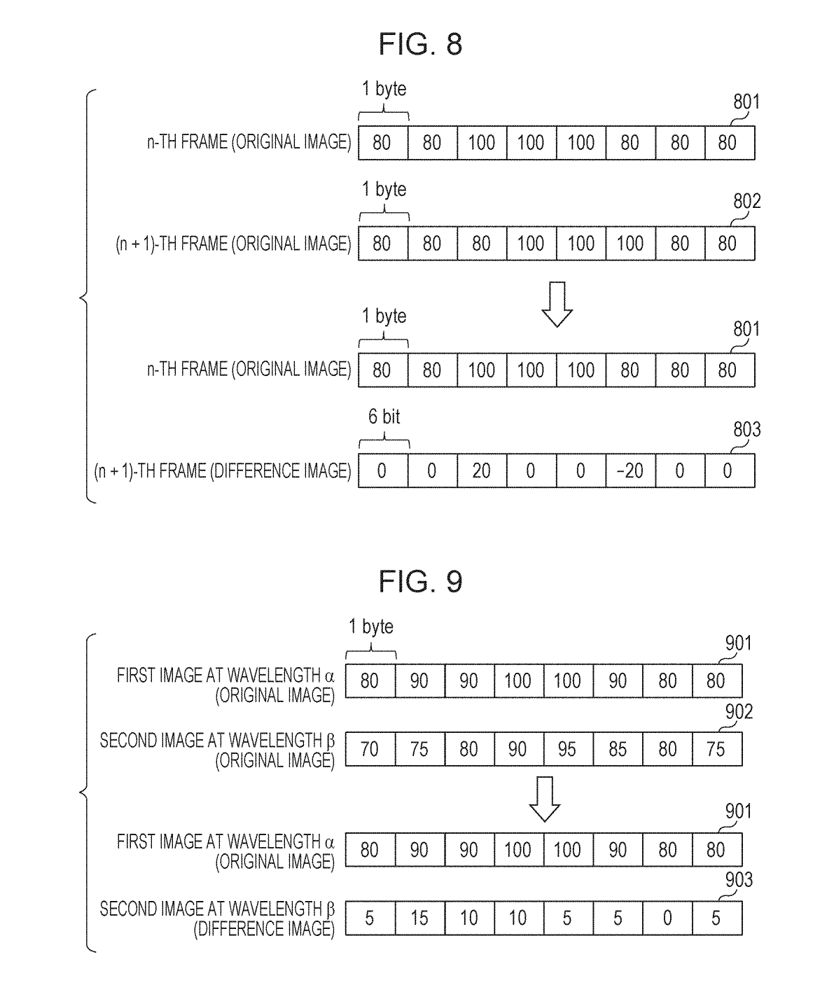

FIG. 8 illustrates an example of the compression method performed by the image processing unit 303 with respect to the ultrasound image or the photoacoustic image. For example, the image processing unit 303 generates the compressed image (compressed data) on the basis of a state in which the images between the adjacent frames which constitute the moving picture are similar to each other. The original image 801 indicates an arrangement of the pixel values of the original image in the n-th frame, and the original image 802 indicates an arrangement of the pixel values of the original image in the (n+1)-th frame. A compressed image 803 is obtained by compressing the original image 802 in the (n+1)-th frame on the basis of the image data in the n-th frame. The image processing unit 303 obtains a difference between the original image 802 and the original image 801 and generates the compressed image 803 in which the difference is set as the pixel value. Since the (n+1)-th frame and the n-th frame are similar to each other, it is predicted that the pixel value of the compressed image 803 corresponding to the difference between these frames is decreased, and the number of bits for the one pixel of the compressed image 803 can be reduced. The same also applies to the (n+2)-frame and the subsequent frames, so that the data amount of the moving picture can be reduced.

The IOD of the image data compressed by the processing exemplified in FIG. 8 is output together with the information indicating that the compression is performed on the basis of the difference of the image data between the frames and the information for identifying the image data (herein, the image data in the n-th frame) obtained by differentiating the original image. The external apparatus such as the viewer 113 that has obtained the IOD previously obtains the information for decoding the compression data obtained by the above-described compression method. The viewer 113 reads out the information indicating the above-described compression method and the information for identifying the image data in the n-th frame from the IOD and obtains the image data in the n-th frame, so that it is possible to decode the compressed image in the (n+1)-th frame.

FIG. 9 illustrates an example of the compression method performed by the image processing unit 303 with respect to the absorption coefficient image and the oxygen saturation image of the photoacoustic images. Both the absorption coefficient image and the oxygen saturation image are obtained by imaging information of a substance having a particular optical characteristic (absorption coefficient) inside the subject. Specifically, the absorption coefficient image and the oxygen saturation image are obtained by imaging information of hemoglobin. Therefore, the absorption coefficient image and the oxygen saturation image are predicted as similar images on which travelling of the blood vessel is reflected, for example. For example, the image processing unit 303 obtains a difference between the absorption coefficient image at the wavelength α and the absorption coefficient image at the wavelength β to generate the compressed image (compressed data). An original image 901 is the absorption coefficient image at the wavelength α, and an original image 902 is the absorption coefficient image at the wavelength β. The image processing unit 303 obtains a difference between the original image 901 and the original image 902 and generates a compressed image 903 of the original image 902. It is predicted that the pixel value of the compressed image corresponding to the difference between the mutually similar images is decreased which are the images obtained by imaging the information of the substance having a particular optical characteristic inside the subject like the absorption coefficient images and the oxygen saturation image. Thus, the number of bits for the one pixel in the compressed image 903 can be reduced.

The IOD of the image data compressed by the processing exemplified in FIG. 9 is output together with the information indicating that the compression is performed on the basis of the difference of the image data at the different wavelengths and the information for identifying the image data (herein, the absorption coefficient image at the wavelength α) obtained by differentiating the original image. The external apparatus such as the viewer 113 that has obtained the IOD previously obtains the information for decoding the compression data obtained by the above-described compression method. The viewer 113 reads out the information indicating the above-described compression method and the information for identifying the absorption coefficient image at the wavelength α from the IOD and obtains the absorption coefficient image at the wavelength α, so that it is possible to decode the compressed image of the absorption coefficient image at the wavelength β.

The output control unit 304 controls the image processing unit 303 to generate the compression data by the compression method in accordance with the type of the medical image. For example, it is conceivable that a gradation change is not preferably applied to the B-mode image that plentifully includes the information related to the mode inside the subject in some cases, and it is estimated that a region where the particular pixel value is continuous hardly exists. In addition, in the case of an image in which information of a particular site in the subject becomes a center like an elastography image or a Doppler image, for example, it is conceivable that the pixel where the pixel value is zero is continuous exists. Therefore, in the case of an B-mode image in which the type of the ultrasound image is represented by a contrasting density, for example, the output control unit 304 may control the image processing unit 303 such that the compression data is generated by reducing the information amount of the high frequency component. In a case where the type of the ultrasound image is an elastography image or a Doppler image, for example, the output control unit 304 may control the image processing unit 303 such that the compression data is generated on the basis of the continuation of the particular pixel value.

The output control unit 304 also controls the image processing unit 303 such that the compression processing is performed in accordance with the type with respect to the type of each of the photoacoustic data. For example, the initial sound pressure data is the image data used for generating the image data of another type, and the compressed is not performed or the lossless compression is performed. For example, the output control unit 304 controls the image processing unit 303 such that the lossless compression is performed with respect to the initial sound pressure data among the plural pieces of the photoacoustic data, and a compression method other than the lossless compression (such as, for example, a method having a higher compression rate than the lossless compression) is performed with respect to the photoacoustic data other than the initial sound pressure data. As an alternative to the above-described configuration, the output control unit 304 may vary the compression rate in accordance with the type of the photoacoustic data. For example, the compression rate of the compression method applied to the initial sound pressure data may be set to be lower than the compression rate of the compression method applied to the photoacoustic data other than the initial sound pressure data. In a case where the absorption coefficient images at the plurality of different wavelengths and the oxygen saturation are included among the types of the photoacoustic data output to the external apparatus, the compression processing based on the difference between the wavelengths may be performed by using a similarity of those.

In step S604, the output control unit 304 performs the association between the images included in the image data 503. The output control unit 304 specifies the corresponding image data by using the correspondence information 507.

In step S605, the output control unit 304 obtains the IOD. The output control unit 304 may also store the pieces of image data associated in step S604 in the same IOD. In the above-described case, the output control unit 304 may store the plural pieces of image data in multi-frames. In addition, the output control unit 304 may the plural pieces of image data in a mode pursuant to a grayscale softcopy presentation state (GSPS) or a colorscale softcopy presentation state (CSPS). The output control unit 304 may store the plural pieces of image data as the multi-frames in the single IOD. The output control unit 304 may generate the respective pieces of image data as the different IODs and include the information for identifying the IODs of the image data corresponding to the auxiliary information of the mutual IODs.

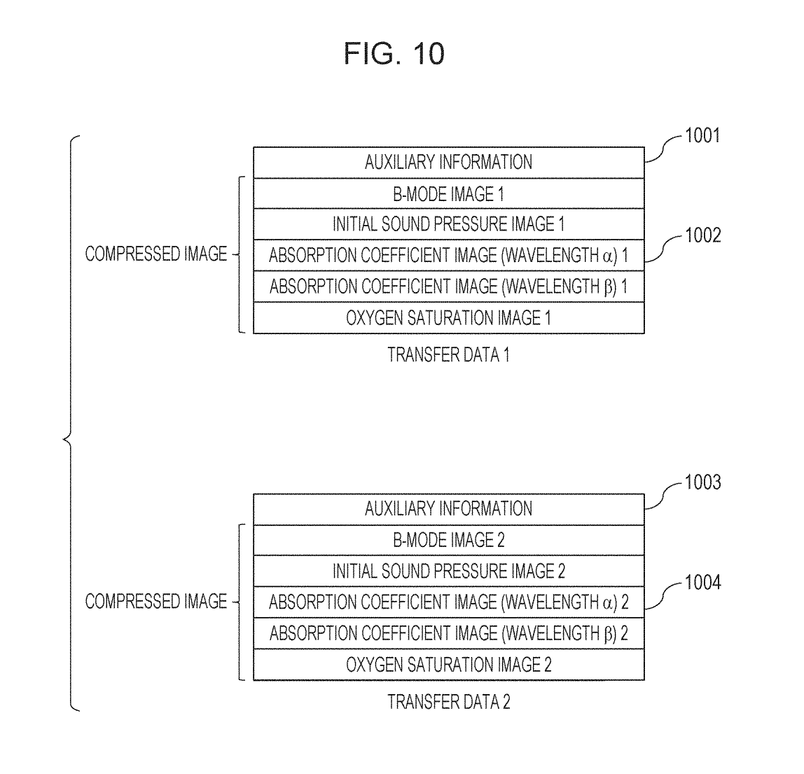

FIG. 10 illustrates an example of the IOD. In the example of FIG. 10, the associated pieces of image data are stored in the single IOD as described in the correspondence information 507 of FIG. 5. Transfer data (IOD) 1 includes auxiliary information 1001 and image data 1002. Transfer data (IOD) 2 includes auxiliary information 1003 and image data 1004. The auxiliary information 1001 and the auxiliary information 1003 may include part or all of the auxiliary information 502 illustrated in FIG. 5. The image data 1002 and the image data 1004 are the compressed images (compressed data) compressed by the above-described processing. All the pieces of the image data included in the IOD are compressed in the example illustrated in FIG. 10, but part of the image data may be compressed. For example, the user obtains the IOD from the information processing apparatus 107 or the PACS 112 via the viewer 113. The viewer 113 can previously obtain a code table. In addition, in a case where the information for decoding the compression data is described in the IOD, the viewer 113 can read out the information for the decoding. With this configuration, the viewer 113 can decode the compression data to be displayed on the display unit (not illustrated) of the viewer 113.

With the configuration according to the first embodiment, the ultrasound image and the photoacoustic image (photoacoustic data) is compressed in accordance with the type and output to the external apparatus such as the PACS 112. With this configuration, the capacity related to the communication of the image data or the saving is reduced.

### Second Embodiment

According to a second embodiment, a case will be described as an example where the type of the image data to be output by the user to the external apparatus is selected, and the compression method can be set.

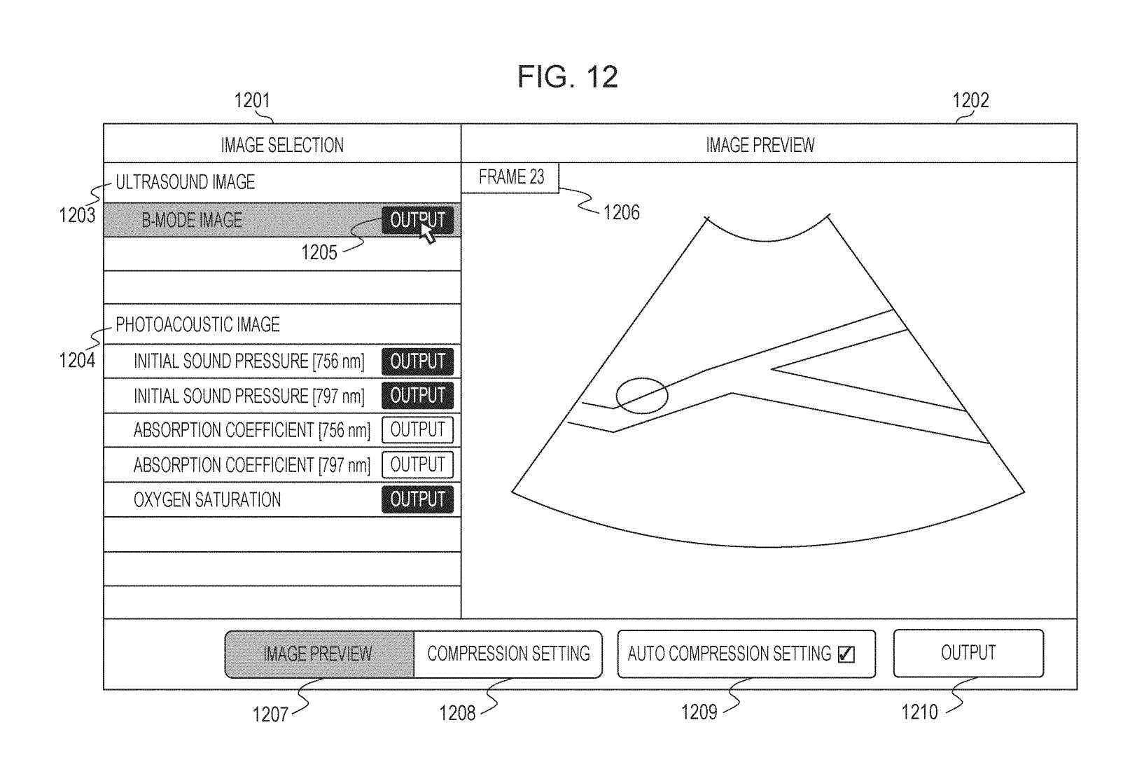

FIG. 12 illustrates an example of the user interface for specifying the type of the image data to be output by the user to the external apparatus. A list of types that can be specified is displayed in a column 1201. In the column 1201, respective types of the ultrasound image are displayed in a region 1203, and respective types of the photoacoustic data are displayed in a region 1204. The user can specify so as to output image data of an arbitrary type to the external apparatus by an operation input with respect to an output button 1205. In the column 1201, the images of the types obtained in step S402 and step S403 illustrated in FIG. 4 are displayed. The information specified by the user via the output button 1205 is stored in the RAM 203 as the information indicating whether or not the output related to the image data of the respective types can be output.

An item in a selected state is displayed so as to be distinguishable from an item that is not in the selected state in the column 1201. In the example illustrated in FIG. 12, the item in the selected state is displayed in a different background color from the other item. When an image preview 1207 is in the selected state, the image data of the type in the selected state in the column 1201 is displayed in a display region 1202. A frame number of the image data displayed in the display region 1202 for the image is displayed in a region 1206.

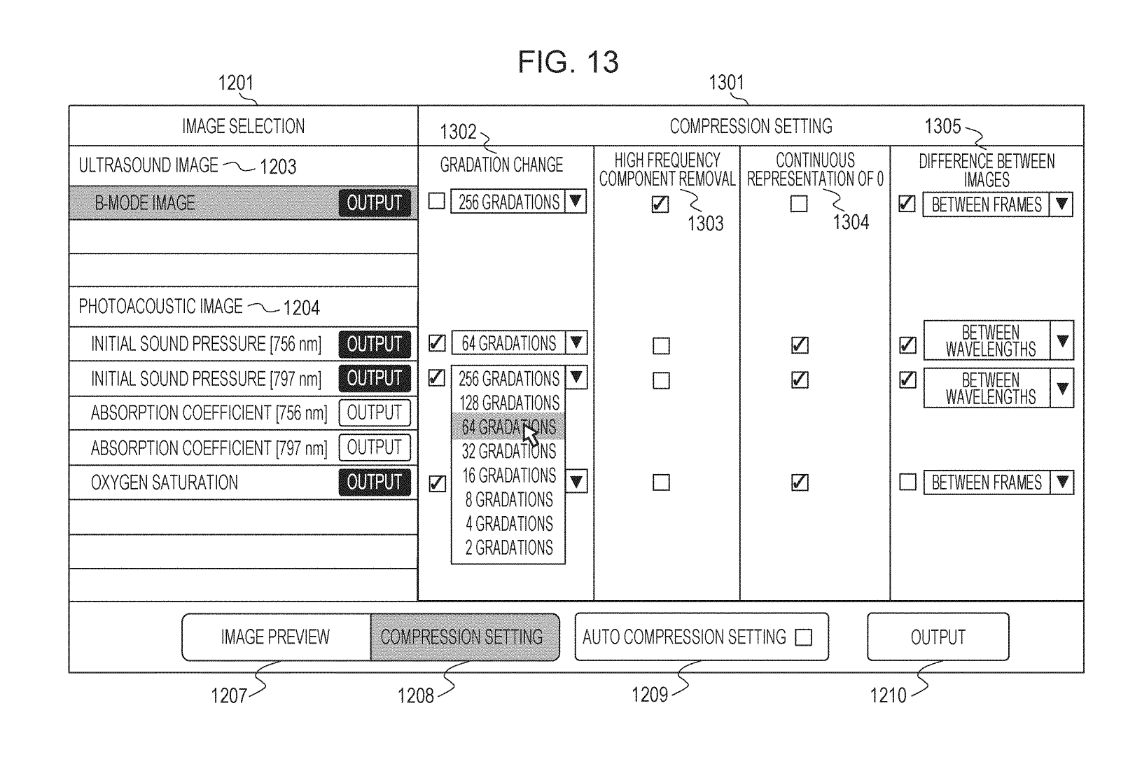

When a compression setting 1208 is selected, the setting screen illustrated in FIG. 13 is displayed on the display unit 109. When an output button 1210 is pressed, the information instructed via the output button 1205 is confirmed.

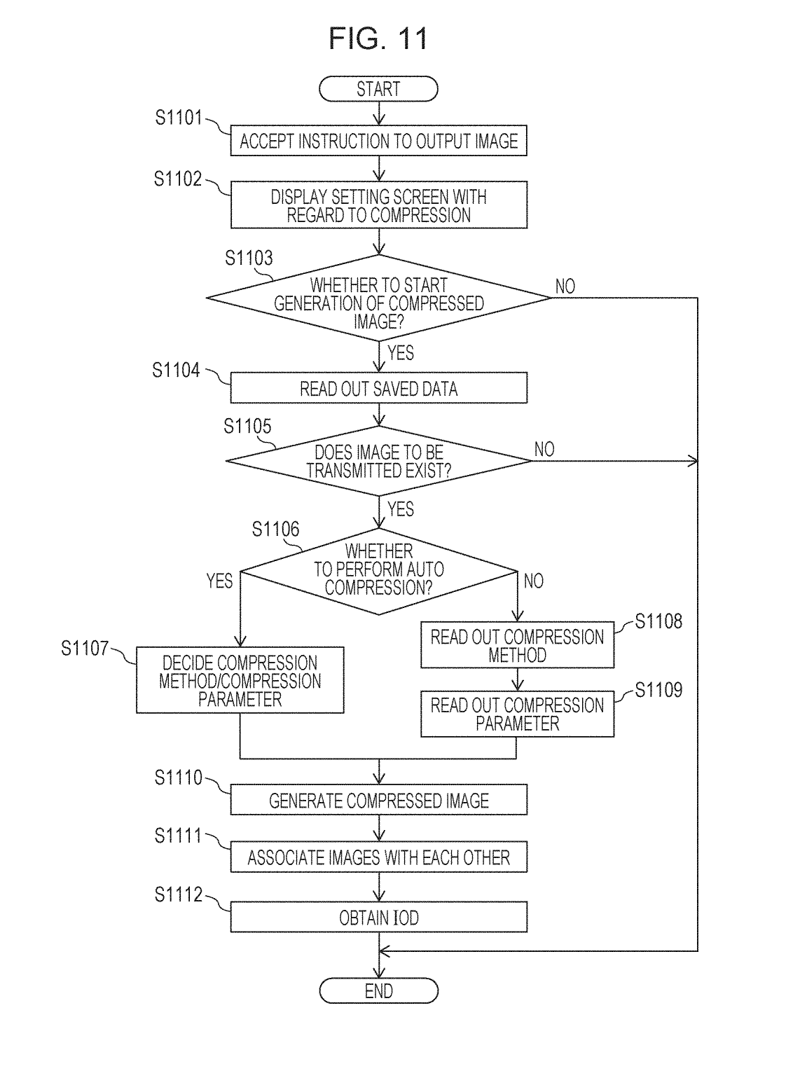

FIG. 11 is a flow chart illustrating an example of processing for compressing the image data to be output on the basis of the specification of the user. The processing will be described with reference to FIG. 12 and FIG. 13 as needed. The processing illustrated in FIG. 11 is performed as a sub routine of step S409 illustrated in FIG. 4, for example. In the following processing, unless specifically stated, the main body that realizes the respective processes is the CPU 201 or the GPU.

In step S1101, the output control unit 304 is the image data selected by the user via the user interface in accordance with the operation input of the user. As an alternative to the above-described configuration, the image data set as the target where the compression data is generated may be selected on the basis of a predetermined setting.

In step S1102, the display control unit 306 causes the display unit 109 to display a setting screen 1301. When the compression setting 1208 illustrated in FIG. 12 is pressed, the display control unit 306 displays the setting screen illustrated in FIG. 13.

FIG. 13 illustrates an example of the setting screen. The column 1201, the region 1203, and the region 1204 are similar to those illustrated in FIG. 12. A region 1302 is a region for inputting an instruction related to the compression processing of the gradation change, and when a check box is set as ON, the gradation change with respect to the image data of the type where the output button is pressed in the column 1201 is enabled. A region 1303 is a region for inputting an instruction related to the compression processing for removing the high frequency component, and when the check box is set as ON, the high frequency component removal with respect to the image data of the type where the output button is pressed in the column 1201 is enabled. A region 1304 is a region for inputting an instruction related to the compression processing using a run length in which 0 is continuous, and when the check box is set as ON, the compression processing is enabled with respect to the image data of the type where the output button is pressed in the column 1201. A region 1305 is a region for inputting the instruction related to the compression processing using the difference between the images, and when the check box is set as ON, the compression processing is enabled with respect to the image data of the type where the output button is pressed in the column 1201. It is possible to select whether the difference between the images is obtained between the frames of the moving picture or between the images obtained at the different wavelengths in the region 1305.

In step S1103, the output control unit 304 determines whether or not the generation of the compression data is to be started. The output control unit 304 determines that the generation of the compression data is started in accordance with the press of the output button 1210 illustrated in FIG. 12.

In step S1104, the output control unit 304 reads out the saved data 501 from the storage device 204.

In step S1105, the output control unit 304 reads out the information as to whether the output of the image data included in the saved data 501 can be performed or not from the RAM 203. As described above, the information as to whether the output can be performed or not is stored in the RAM 203 on the basis of the instruction with respect to the output button 1205 illustrated in FIG. 12.

In step S1106, the output control unit 304 determines whether or not it is set that the compression processing of the image data of the type to be output is to be automatically performed. When the auto compression setting 1209 in FIG. 12 and FIG. 13 is set as ON, it is set that the compression processing is to be automatically performed, and the information is stored in the RAM 203. The output control unit 304 reads out the information of the setting as to whether or not the compression processing is to be automatically performed from the RAM 203. In a case where it is set that the compression processing is to be automatically performed, the flow proceeds to step S1107, and in a case where it is set that the compression processing is not to be automatically performed, the flow proceeds to step S1108.

In step S1107, the output control unit 304 obtains the information related to the previously set compression processing. For example, the output control unit 304 performs the compression processing by the high frequency component removal with respect to the B-mode image among the ultrasound images. The gradation change is not preferably applied to the B-mode image that plentifully includes the information related to the mode inside the subject, and it is predicted that a region where the particular pixel value is continuous hardly exists. Therefore, the output control unit 304 performs the compression processing for removing the high frequency component with respect to the B-mode image.

The output control unit 304 performs the compression processing in accordance with the type with respect to the type of each of the photoacoustic data. For example, the initial sound pressure data is the image data used for generating the image data of the other type, and the compressed is not performed or the lossless compression is performed. In a case where the absorption coefficient images at the plurality of different wavelengths among the types specified to be output in the output buttons 1205 of FIG. 12 and the oxygen saturation are included, the compression processing at the difference between the wavelengths is performed by using the similarity of those images.

The output control unit 304 may further select the compression rate or the compression method so as to fulfill the maximum capacity in the single IOD which is defined by the DICOM standard. In this case, the output control unit 304 may decrease the compression rate of the image such as the oxygen saturation used in the diagnosis than that of the other types.

In step S1108, the output control unit 304 obtains the information related to the compression method specified by the user via the setting screen 1301 of FIG. 13.

In step S1109, the output control unit 304 reads out the information of the parameter corresponding to the compression method obtained in step S1108 from the RAM 203.

In step S1110, the output control unit 304 controls the image processing unit 303 to generate the compression data. The image processing unit 303 compresses the image data on the basis of the information of the parameter obtained in step S1107 or step S1109.

In step S1111, the output control unit 304 performs the association between the images included in the image data 503. The output control unit 304 specifies the corresponding image data by using the correspondence information 507.

In step S1112, the output control unit 304 obtains the IOD. The output control unit 304 may store the image data associated in step S1111 in the same IOD. In the above-described case, the output control unit 304 may store the plural pieces of image data in multi-frames. In addition, as described above according to the first embodiment, the output control unit 304 may store the plural pieces of image data in a mode pursuant to the GSPS or the CSPS. The output control unit 304 may store the plural pieces of image data as the multi-frames in the single IOD. The output control unit 304 may generate the respective pieces of image data as the different IODs and include the information for identifying the IODs of the image data corresponding to the auxiliary information of the mutual IODs.

With the configuration according to the second embodiment, the information processing apparatus 107 can compress the image data of the arbitrary type specified by the user by the compression processing in accordance with the type to be output to the external apparatus.

### Modified Examples

The case has been described as an example where the saved data 501 saved up to step S406 illustrated in FIG. 4 is read out to generate the compression data, but the present invention is not limited to this. For example, the compression processing may be performed before the image data is stored in the saved data 501, and the compression data may be stored in the saved data 501.

The present invention is also realized by processing in which a program that realizes one or more functions of the above-described embodiments is supplied to a system or an apparatus via a network or a storage medium, and one or more processors in a computer of the system or the apparatus reads out the program to be executed. In addition, the present invention is realized by a circuit (for example, an ASIC) that realizes one or more functions.

The information processing apparatus according to the above-described respective embodiments may be realized as a standalone apparatus and may also adopt a mode in which a plurality of apparatuses are combined so as to be mutually communicable to execute the above-described processing, both of which are also included in the embodiments of the present invention. A common server apparatus or a server group may also execute the above-described processing. It is sufficient when the plurality of apparatuses constituting the information processing apparatus and the information processing system are communicable at a predetermined communication rate and are not required to be located in the same facility or in the same country.

The embodiments of the present invention include a mode in which a software program for realizing the functions of the above-described embodiments is supplied to a system or an apparatus, and a computer of the system or the apparatus reads out and executes a code of the supplied the program.

Therefore, in order that the computer realizes the processing according to the embodiments, a program code itself to be installed in the computer is also one of the embodiments of the present invention. In addition, an operating system (OS) or the like running on the computer performs part or all of the actual processes on the basis of an instruction included in the program read out by the computer, and the functions of the above-described embodiments may also be realized by the processing.

A mode obtained by appropriately combining the above-described embodiments with each other is also included in the embodiments of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus comprising:
an obtaining unit configured to obtain photoacoustic data generated on a basis of a photoacoustic signal obtained by irradiating a subject with light;
a compression unit configured to obtain compressed data obtained by compressing the photoacoustic data in accordance with a type of the photoacoustic data obtained on a basis of the photoacoustic signal; and
an output unit configured to output the compressed data to an external apparatus.

2. The information processing apparatus according to claim 1, wherein the compression unit applies different compression methods in accordance with types of the photoacoustic data.

3. The information processing apparatus according to claim 2, wherein the compression method applied to initial sound pressure data and the compression method applied to the photoacoustic data other than the initial sound pressure data are different from each other.

4. The information processing apparatus according to claim 3, wherein the compression method applied to the initial sound pressure data is lossless compression.

5. The information processing apparatus according to claim 1, wherein the compression unit performs compression at a different compression rate in accordance with the type of the photoacoustic data.

6. The information processing apparatus according to claim 5, wherein the compression rate with respect to initial sound pressure data and the compression rate with respect to the photoacoustic data other than the initial sound pressure data are different from each other.

7. The information processing apparatus according to claim 6, wherein the compression rate with respect to the initial sound pressure data is lower than the compression rate with respect to the photoacoustic data other than the initial sound pressure data.

8. The information processing apparatus according to claim 1, wherein the output unit outputs the compressed data and information indicating a decoding method to the external apparatus as a single information object.

9. An information processing apparatus comprising:
an obtaining unit configured to obtain data by irradiating a subject with light;
a compression unit configured to obtain compressed data obtained by compressing the data obtained by the obtaining unit; and
an output unit configured to output the compressed data and a decoding method for decoding the compressed data as a single information object to an external apparatus.

10. The information processing apparatus according to claim 8, wherein the information object is an object based on a Digital Imaging and Communications in Medicine (DICOM) standard.

11. An information processing method comprising:
obtaining photoacoustic data generated on a basis of a photoacoustic signal obtained by irradiating a subject with light;
obtaining compressed data obtained by compressing the photoacoustic data in accordance with a type of the photoacoustic data obtained on a basis of the photoacoustic signal; and
outputting the compressed data to an external apparatus.

12. A non-transitory computer-readable medium storing a program for causing a computer to execute the information processing method according to claim 11.
